(19) United States
(12) Patent Application Publication
(10) Pub. No.: US 2024/0423746 A1
(43) Pub. Date: Dec. 26, 2024

(54) RADIATION COMPATIBLE EXPANDER FOR BREAST RECONSTRUCTION

(71) Applicant: University of Florida Research Foundation, Incorporated, Gainesville, FL (US)

(72) Inventors: Julie BRADLEY, Jacksonville, FL (US); John D. MURRAY, Jacksonville, FL (US); Michele V. MANUEL, Jacksonville, FL (US); Wesley Emmett BOLCH, Jacksonville, FL (US); Xiaoying LING, Jacksonville, FL (US)

(21) Appl. No.: 18/686,633

(22) PCT Filed: Aug. 26, 2022
(86) PCT No.: PCT/US2022/041653

§ 371 Date: Feb. 26, 2024

Related U.S. Application Data
(60) Provisional application No. 63/237,471, filed on Aug. 26, 2021.

(51) Int. Cl. A61B 90/00; A61F 2/12

(52) U.S. Cl. CPC A61B 90/02; A61F 2/12

(57) ABSTRACT

Provided is a system for expanding breast tissue using a breast tissue expander comprising a pouch for holding fluid and a port for injecting fluid into the pouch. wherein the port is comprised of a low-Z material. Examples of low-Z material include aluminum, magnesium, combinations thereof, and alloys thereof. Also disclosed is a system comprising a breast tissue expander and an inductance meter.

BACKGROUND

Breast cancer is the most common non-skin cancer affecting women in the US, with greater than 250,000 women diagnosed in the US annually. In 2016, 33% of women with Stage I-II breast cancer and 68% of women with Stage III underwent mastectomy for treatment of their breast cancer. Between 1998 and 2008, immediate breast reconstruction rates increased by 5% per year and by 2002 expander reconstruction comprised the most common type of breast reconstruction after mastectomy. This procedure includes placement of a tissue expander underneath or on top of the pectoralis major muscle after the breast has been removed. In such expander based postmastectomy breast reconstruction, the expander stretches the remaining mastectomy skin and subcutaneous tissue and creates a “house” for a subsequently exchanged permanent breast implant. The expander undergoes expansion when sterile saline is transcutaneously injected into a port which is located in the anterior wall of the expander. The expansion process typically relies on three to six infusions over a period of several weeks before the expander is removed and the implant is placed.

Postoperatively, the patient presents weekly to the plastic surgeon for injection of saline through the port of the expander to stretch the tissue and create the desired reconstructed breast size and shape. The plastic surgeon uses a hand-held magnet to locate the injection site of the expander, which is denoted by the expander's magnet. FIG. 1A is an example of a known ex vivo expander with an injection port accessed by a needle to allow for saline injection. The dark area underlying the needle is the injection port, which contains the cup and the magnet. FIG. 1B is an example of a known breast tissue expander, having a silicone outer shell and a valve or port comprising a central neodymium magnet set into the base of a titanium plate with a slight rim. FIG. 1C is an example of the magnetic valve or port also shown in FIG. 1B. The injection site typically has a thin stainless-steel or titanium cup that prevents the needle from passing too deep and puncturing the expander. Unfortunately, a stainless-steel cup and a neodymium (Nd) magnet are both high atomic number (Z) materials that interfere with both radiation therapy and imaging. FIG. 1D is an X-ray showing the breast tissue expander and the port from FIGS. 1B and 1C after implantation into a patient.

High-Z materials (defined in detail hereinafter) create an artifact on treatment planning computed tomography (CT) scan images, regardless of the type of beam selected for treatment. FIG. 2A is an example of a prior art axial CT slice, focusing on soft tissue, of a patient with a left reconstructed breast with a tissue expander in place. FIG. 2B is an example of a prior art axial CT slice, focusing on bone, of a patient with a left reconstructed breast with a tissue expander in place. As can be seen, the high-Z materials, such as the magnet, creates an artifact (denoted by arrows) that affects a significant amount of the radiation target volume and extends well beyond the magnet itself. This streak artifact (which is attributed to enhanced beam hardening and scatter as the x-ray field enters the high-Z component of the device) must then be overridden and assigned the appropriate tissue or saline density. In CT, the term artifact is applied to any systematic discrepancy between the CT Hounsfield unit (HU) numbers in the reconstructed image and the true attenuation coefficients of the object. The presence of high-Z objects in the patient can lead to severe streaking artifacts. They occur because the density of the metal is beyond the normal range that can be handled by the computer, resulting in incomplete attenuation profiles. The artifact can result in significant dose perturbation for proton therapy, as the accuracy of the modelled dose distribution in the treatment planning system (TPS) is very sensitive to the accuracy of the assignment of the proton relative stopping power (RSP) along the beam path. A lookup table is used to convert the CT HU number to RSP. Therefore, the artifact-free CT is a key for an accurate modelling of the dose distribution for proton therapy. A variety of interpolation techniques/metal artifact reduction software has been developed to reduce the streaking artifact. While the metal artifact reduction software does a reasonable job in reducing artifacts caused by metals with relatively low-Z, atomic numbers, such as titanium, it is useless in reducing the artifact caused by many high-Z magnetic materials such as the Nd used in the current commercial breast tissue expanders.

Furthermore, the magnet has a much higher relative stopping power (>5 times) than the surrounding tissue. The interaction of radiation beams with tissue/medium are affected by the tissue/medium densities through which they pass. FIG. 3A is an example showing imaging associated with a double scatter proton therapy, illustrating that underdosing can occur posterior to the port of a prior art expander if the relative stopping power of the prior art expander is underestimated. FIG. 3B is an example showing imaging associated with a double scatter proton therapy, illustrating that overshooting can occur posteriorly above and below the port of a prior art expander due to smearing if the stopping power of the prior art expander is appropriate for the metal component. Particle radiation (protons, electrons, carbon-ions) are more subject to dose perturbation than photon beams, though photon beams do show degradation of up to 20% due to the metal component of the tissue expander. For pencil beam scanning proton irradiation, techniques have been developed to manage the dose perturbation or avoid the metal of the expander using a “treat around” technique. While acceptable, these techniques are suboptimal due to increased dose distribution heterogeneity including cold spots in the target region and hot spots in the adjacent organs at risk as well as potential movement of the metal component of the expander. If the metal moves between the radiation planning session and the treatment, there is a risk of significantly underdosing target tissue and overdosing adjacent healthy tissue. The use of proton therapy is increasing worldwide. The number of protons centers in the US has grown from five in 2006 to thirty-one in 2020. Globally, there are 102 proton centers. The first carbon ion center in the US has been announced for development by Mayo Clinic in Jacksonville, Florida, and it will join 13 carbon-ion centers worldwide. Thus, the need for a radiation compatible tissue expander is increasing exponentially.

Another complication with the use of magnetic materials is in magnetic resonance imaging (MRI) compatibility. Currently, the presence of a breast tissue expander is a contraindication to MRI. Often patients with breast cancer would benefit from MRI in the post-surgical period to assess for locoregional or distant disease progression, but due to the presence of a tissue expander with magnetic components, CT is obtained instead, or the MRI is deferred until after the tissue expander has been replaced with an implant.

While expander-based breast reconstruction is the most common method of breast reconstruction in the United States, accounting for about 70,000 cases in 2018, it is also one of the oldest. The first published description of the technique was in 1982. Since then, the fundamental design of expanders, to include the port, has not significantly changed. Though the port design has not changed, technology which detects and treats primary breast cancer and its metastases has improved significantly, leading to improved cancer specific survival. Such technology includes high tesla MRI and adjuvant proton beam radiotherapy to the remaining mastectomy tissues. However, due to the materials used in the port, patients with a breast expander cannot undergo MRI. Additionally, as the port materials create areas of proton beam perturbation, or beam path irregularity within the tissues, respective areas of under treatment are possible, largely due to their high atomic numbers (high-Z). MRI and proton beams are known to be particularly sensitive to the electrical disturbances offered by high-Z metals. A further consideration is the streak artifact that occurs on the CT images (independent of radiation modality). The artifact can make target delineation difficult through distortion of the anatomy in the region of the artifact. In addition, for proton planning, the artifact is contoured and reassigned to the actual CT density.

Accordingly, there is a need for a new expander port that offers MRI and proton beam compatibility, uses materials with high corrosion resistance, and, in cases where a magnet is not integrated into the port design, introduces an alternative hand-held method to clinically locate the port.

The discussion of shortcomings and needs existing in the field prior to the present invention is in no way an admission that such shortcomings and needs were recognized by those skilled in the art prior to the present disclosure.

### BRIEF DESCRIPTION OF THE FIGURES

Many aspects of this disclosure can be better understood with reference to the following figures.

FIG. 1A is an example of a known ex vivo expander with an injection port accessed by a needle to allow for saline injection.

FIG. 1B is an example of a known breast tissue expander, having a silicone outer shell and a valve or port comprising a central neodymium magnet set into the base of a titanium plate with a slight rim.

FIG. 1C is an example of the magnetic valve or port also shown in FIG. 1B.

FIG. 1D is an X-ray showing the breast tissue expander and the port from FIGS. 1B and 1C after implantation into a patient.

FIG. 2A is an example of a prior art axial CT slice, focusing on soft tissue, of a patient with a left reconstructed breast with a tissue expander in place.

FIG. 2B is an example of a prior art axial CT slice, focusing on bone, of a patient with a left reconstructed breast with a tissue expander in place.

FIG. 3A is an example showing imaging associated with a double scatter proton therapy, illustrating that underdosing can occur posterior to the port of a prior art expander if the stopping power of the prior art expander is underestimated.

FIG. 3B is an example showing imaging associated with a double scatter proton therapy, illustrating that overshooting can occur posteriorly above and below the port of a prior art expander due to smearing if the stopping power of the prior art expander is appropriate for the metal component.

FIG. 4A is an example of a known adult female polygon mesh phantom from the International Commission on Radiological Protection (ICRP) which is useful for testing purposes as a virtual patient.

FIG. 4B is an example of a cross-section of the known adult female polygon mesh phantom according to FIG. 4A.

FIG. 5A is an example, illustrating a CT image across the solid water phantom 1 cm below a prior art tissue expander including a Nd magnet.

FIG. 5B is an example according to various embodiments, illustrating a HU profile across the solid water phantom 1 cm below a prior art tissue expander including a Nd magnet.

FIG. 6A is an example, illustrating a CT image across the solid water phantom 1 cm below a tissue expander according to various embodiments that comprises a Mg alloy.

FIG. 6B is an example, illustrating a HU profile across the solid water phantom 1 cm below a tissue expander according to various embodiments that comprises a Mg alloy.

FIG. 7A is an example, illustrating a CT image across the solid water phantom 1 cm below a tissue expander according to various embodiments that comprises no high-Z material in the field.

FIG. 7B is an example according to various embodiments, illustrating a HU profile across the solid water phantom 1 cm below a tissue expander according to various embodiments that comprises no high-Z material in the field, which provides the ground truth of the HU number of the solid water phantom.

FIG. 8 shows a side view of a breast tissue expander embodiment.

FIG. 9 shows a schematic view of a portable detector for detection of a port according to embodiments disclosed herein.

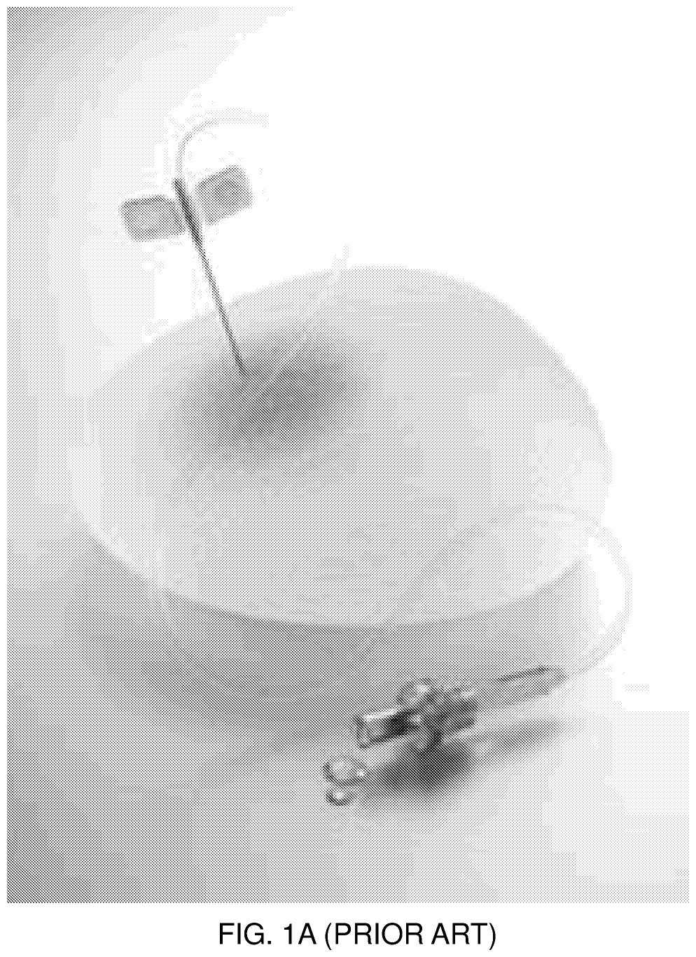

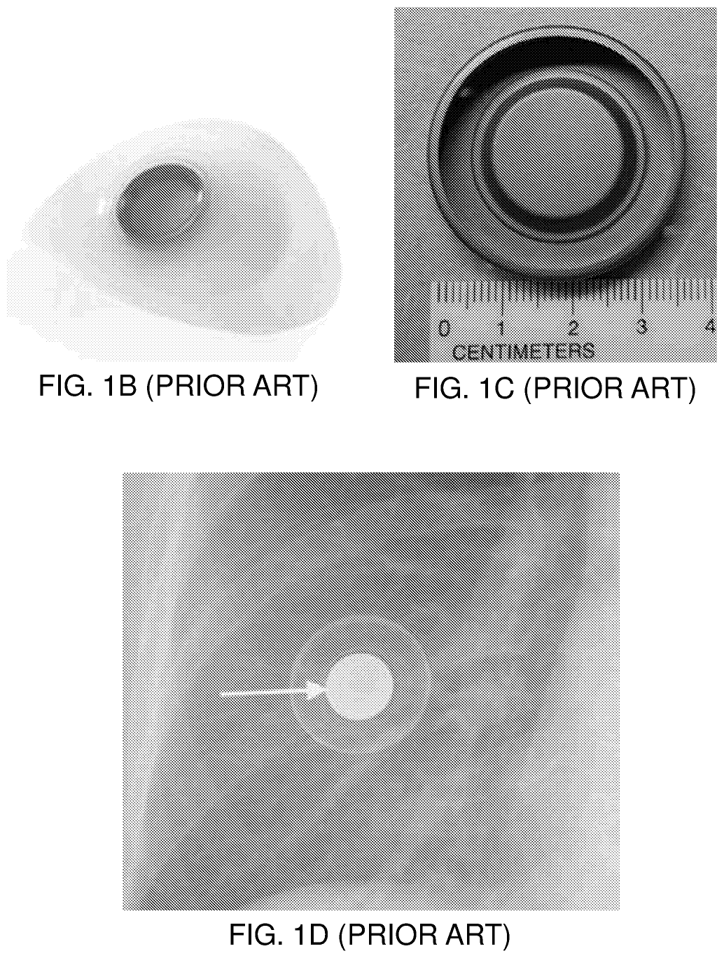

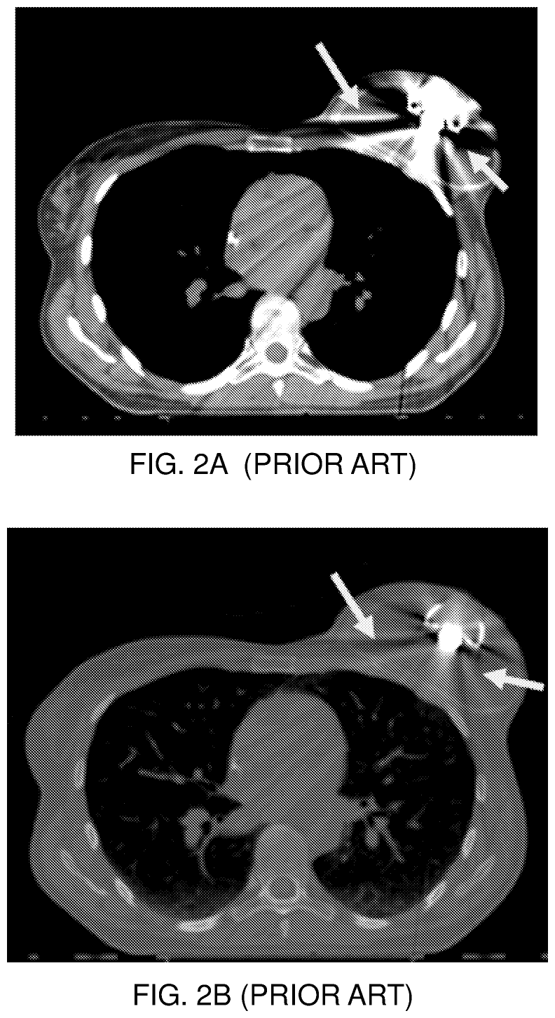

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

In this specification and in the claims that follow, reference will be made to a number of terms that shall be defined to have the following meanings unless a contrary intention is apparent.

Unless otherwise specified, all percentages indicating the amount of a component in a composition represent a percent by weight of the component based on the total weight of the composition.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As used herein, the term “high-Z” materials refers to chemical elements with a high atomic number (Z) of protons in the nucleus, to combinations of such chemical elements, or to materials containing such elements in sufficient amounts to interfere with medical imaging or medical treatment. In this context, “medical imaging” includes but is not limited to CT and MRI; and “medical treatment” includes but is not limited to radiation therapy. A “high atomic number” is typically any element with an atomic number greater than or equal to the atomic number of scandium (Sc). Common examples of “high-Z” elements include, but are not limited to chromium (Cr), vanadium (V), iron (Fe), and neodymium (Nd). Stainless steel is another example of a high-z material. Stainless steel typically contains a minimum of approximately 11% chromium. Different types of stainless steel include the elements carbon, nitrogen, aluminum, silicon, sulfur, nickel, copper, selenium, niobium, manganese, chromium, vanadium, and molybdenum.

As used herein, the term “low-Z” materials refers to chemical elements with a low atomic number (Z) of protons in the nucleus, to combinations of such chemical elements, or to materials containing high-Z materials in insufficient amounts to interfere with medical imaging or medical treatment. In this context, “medical imaging” includes but is not limited to CT and MRI; and “medical treatment” includes but is not limited to radiation therapy. A “low atomic number” is typically any element with an atomic number less than the atomic number of scandium (Sc).

As used herein, the term “nonferrous” refers to metals or alloys that do not contain iron (ferrite) in sufficient amounts to interfere with medical imaging or medical treatment. In this context, “medical imaging” includes but is not limited to CT and MRI; and “medical treatment” includes but is not limited to radiation therapy. In general, nonferrous metals and alloys are “nonmagnetic,” with the exception of pure nickel, which is slightly magnetic.

As used herein, the term “disposed on” refers to a positional state indicating that one object or material is arranged in a position adjacent to the position of another object or material. The term does not require or exclude the presence of intervening objects, materials, or layers.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

### General Discussion

Various embodiments relate to a tissue expander using a metal with a low atomic number that is radiotherapy and MRI compatible. Some embodiments employ a low-Z material and different localization method of the injection site in order to improve the dose heterogeneity and target coverage in breast cancer radiotherapy for women with expanders. Low density metals cannot be detected by a magnet, so various embodiments use an Eddy current (metal detector) instead. This tissue expander according to various embodiments simultaneously allows optimal cosmesis, radiotherapy, and diagnostic imaging.

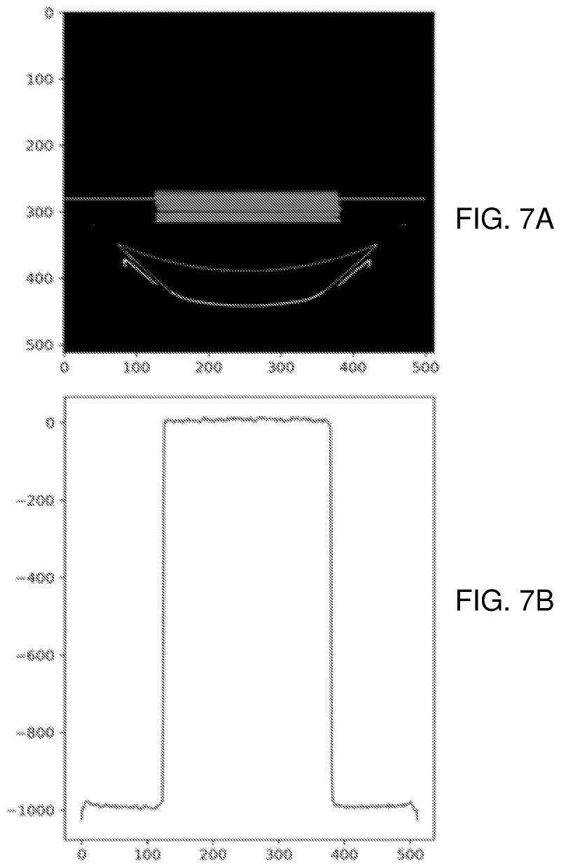

FIG. 8 illustrates one example of a tissue expander 10 configured for breast tissue expansion. The tissue expander 10 comprises a pouch 12 into which fluid can be injected. The pouch 12 can be comprised of any suitable materials known in the art. In a specific example, the pouch 12 is comprised of silicone. The tissue expander 10 also includes a port 14 for injection of fluid into the pouch 12. As explained herein, the port 14 is comprised of a low-Z material, such as aluminum, magnesium, combinations thereof, or alloys thereof.

Another embodiment pertains to a method for expanding breast tissue using a breast tissue expander comprising a port comprised of low-Z material. The method involves implanting a breast tissue expander into a patient at a target site; locating the port of the breast tissue expander; and injecting fluid into the pouch via the port. The method may further involve locating the port by use of an inductance meter or ultrasound.

Currently, the radiotherapy is undergoing adaptations and workarounds to accommodate the tissue expanders. Various embodiments relate to a breast tissue expander to facilitate optimal radiotherapy that is not hindered by the high-Z, magnetic metallic components present in current tissue expanders on the market. Such embodiments will also permit MRI evaluations for patients.

Various embodiments provide a replacement for the port on a conventional breast tissue expander. The port according to various embodiments may replace the Nd magnet with a nonferrous, non-magnetic, low-Z material to ultimately minimize or avoid any deleterious interactions with the radiation beam and MR imaging.

Replacing the Nd magnet with a nonmagnetic material interferes with the current technique of the surgeon using a magnet to locate the port. Thus, various embodiments provide a method of locating the port by using electrical inductance meter, commonly known as a metal detector, or by using ultrasound. Inductance meters internally have two basic systems, one which sends out an electromagnetic signal that induces an eddy current in a buried metal and this eddy current then itself induces its own electromagnetic signal which is detected by the second system that collects the induced electromagnetic signal from the metal. Since the purpose of the first system is to induce an eddy current, the material that is being detected must be electrically conductive. As conductivity decreases, the resultant induced electromagnetic field is reduced making the object difficult to detect. Various embodiments relate to the use of several non-ferrous, non-magnetic low-Z, high conductivity alloys including aluminum (Al) and magnesium (Mg)-based alloys instead of the clinical standard neodymium magnet and stainless-steel cup system. Alternatively, the low Z magnet port may be localized using ultrasound, as the difference in density between the low Z metal and the adjacent soft tissue or expander saline makes the port readily identifiable on ultrasound.

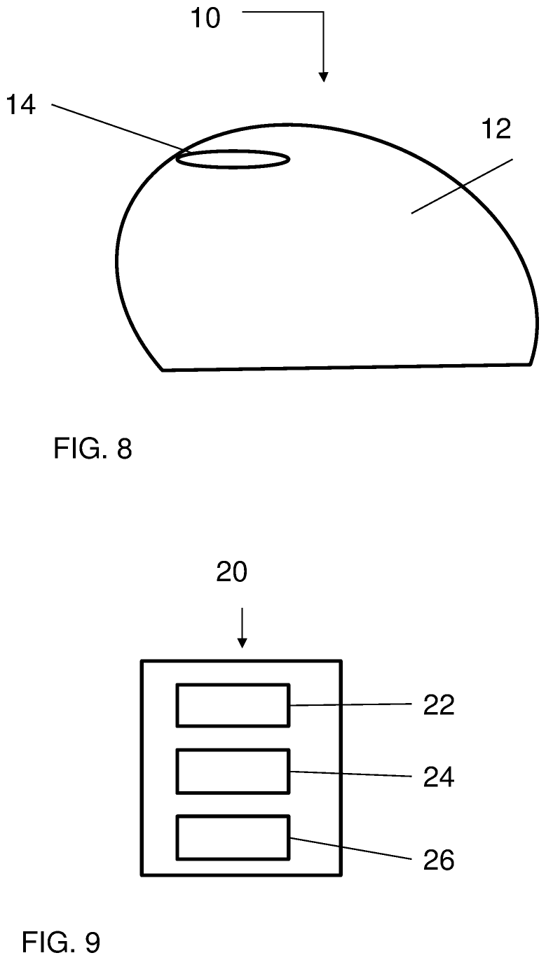

Those skilled in the art, equipped with the teachings herein will appreciate that any number of inductance meters (e.g. metal detectors) known in the art can be adapted to detect the tissue expander port according expander embodiments and methods described herein. See, for example, U.S. Pat. No. 9,155,490. FIG. 9 provides a schematic of an inductance meter for use in locating the port. FIG. 9 shows a portable metal detector 20 that includes a sensor 22, and optionally a signal component 24, and/or a transmitter 26. The sensor 22 is configured to detect metal, and the metal detector 20 can be tuned to detect low-Z materials such as aluminum or magnesium. The transmitter 26 is configured to induce a magnetic field that can generate a voltage as the metal detector 20 is moved across a target site. The signal component 24 can provide an audible, visual, and/or tactile feedback indicating to the user the presence of a metal as the metal detector is moved across a target site.

Use of a low-Z metal in a tissue expander will result in decreased artifact on the planning CT and decreased beam perturbation due to its low relative stopping power, and the low-Z metal will be detectable in tissue by an inductance meter. In a virtual clinical trial of female phantoms of varying breast and body sizes, the proton therapy plans with the redesigned expander may have an average of a 10% decrease in the amount of ipsilateral lung receiving 20 Gy compared to the current commercial expander.

Various embodiments provide a port comprising a low-Z material that minimizes CT artifact, does not affect the radiation beam (specifically proton beam) and can be detected with a hand-held inductance device through soft tissue. Various embodiments provide an expander system with low-Z, highly conductive metal whereby the port can be located with a common metal detector or ultrasound. The ports according to various embodiments may replace the current stainless-steel or titanium cup and Nd magnet with a low-Z alloy cup that does not require a magnet and instead is detected by an inductance meter. The port according to various embodiments offers MRI and proton beam compatibility, uses materials with high corrosion resistance, and provides an alternative hand-held method to clinically locate the port.

According to various embodiments, a low-Z material may be MRI and proton beam compatible. Additionally, various low-Z materials, such as aluminum or magnesium, are also highly corrosion resistant. The port, according to various embodiments may be located using a hand-held device which detects harmless induced electrical currents, termed eddy currents, which both Al and Mg metals and alloys elaborate or by ultrasound. Additionally, in embodiments where the magnet is eliminated, port manufacturing costs may be potentially reduced. With use of this paradigm shifting port design, tens of thousands of patients each year may experience improved cancer detection (with MRI) and improved therapeutic benefit (with, for example, postmastectomy proton beam radiotherapy or photon therapy) while having the reconstructive expander in place, all the while allowing a timely reconstructive endpoint.

Refinement of various embodiments may involve characterizing the proton beam interaction and resultant CT images of low-Z, non-magnetic, puncture resistant alloys relative to the Nd magnet clinical standard.

In this respect it is desirable to focus on characterizing the proton beam interaction and induced CT image artifacts of three different metals, Al, Mg, and Nd magnet in a stainless-steel cup (standard commercial expander design). Specifically, measuring the relative stopping power to assess the level of proton beam interaction.

### Selection of Low-Z Materials

Design criteria for selection of low-Z metal alloys may include non-magnetic, low-Z major alloying elements, susceptibility to detection through use of an inductance meter, commercial availability, and high mechanical performance. Using these factors, the alloys selected may include the Al alloy, Al-6061, and Mg alloy, AZ61. Mechanical and physical properties of these alloys in relation to the conventionally used 316L stainless-steel shell are displayed in Table 1. Additionally, since these are common commercial alloys enabling the procurement of homogeneous metals for experimental trials. The test alloys will be disc-shaped and designed with a puncture strength equivalent to or greater than 316L stainless-steel (thus, compared to the current standard of care stainless-steel cup. Greater strength will enable thinner designs that yield an equivalent strength while minimizing the amount of metal in the device, thus creating less beam perturbation. The low-Z metal alloys may be the same diameter but different thickness to achieve equivalent puncture strength).

TABLE 1
Mechanical and physical properties of metallic alloys of interest.13

| Alloy | Strength (yield) | Z of major alloying element |
|---|---|---|
| Aluminum Alloy (Al-6061) | 280 MPa | 13 |
| Magnesium Alloy (AZ61) | 183 MPa | 12 |
| Stainless-Steel (316L) | 262 MPa | 26 |
| Neodymium (Nd) | — | 60 |

### Quantification of the CT Artifact Created by the Metals

It is possible to perform a CT scan on a solid water phantom to serve as the ground truth that no artifact occurs. Thereafter, it is possible to place the Nd on top of the solid water phantom and obtain a CT scan. Next, the solid water phantom may be scanned with each alloy in Table 1. The artifact may be quantified by obtaining the HU number within the solid water phantom from the CT images and compare between the control (solid water phantom alone), the current approach (Nd) and the proposed approach (each low-Z alloy).

### Measurement of the Range Pull Back of the Low-Z Metals to Obtain Relative Stopping Power

Despite the low-Z property of the proposed metals compared with the currently commercially used high-Z Nd magnet in the tissue expander, the dose perturbation needs to be investigated and accurately modeled. It is possible to measure the relative stopping power of the various alloys listed in Table 1 by measuring the range pull back using Zebra (IBA dosimetry, Germany), a multi-layer ion chamber device. In addition, the depth dose curve of a single spot may be measured with and without the alloys in the beam path, using a Stingray detector (IBA dosimetry, Germany), a large air-vented plane-parallel ionization chamber, for a more accurate measurement. The relative stopping power of the metals may be deducted from the above measurements and it is possible to establish which metal results in minimal beam perturbation.

### Monte Carlo Simulations of Relative Stopping Powers of Tested Metal Alloys

It is possible to validate the results from Aim 1C studies via Monte Carlo radiation transport modeling using the TOPAS code (https://gray.mgh.harvard.edu/research/software/256-topas). TOPAS is a GEANT4-based code specifically designed to perform virtual treatment planning in proton radiotherapy with source term models that are specific to the IBA systems presently in place within UFHPTI. 14-16 Via MC transport modeling, we will assess proton ranges and relative stopping powers of each of the various materials tested. Range pull-back assessments will be compared to those performed using the multi-layer ion chamber studies.

It is, therefore, possible to identify proton dosimetry characteristics of low-Z metals and ability to avoid CT streak artifacts and creation of Monte Carlo simulations of relative stopping power to facilitate treatment planning on virtual patients.

Refinement of various embodiments may further involve designing a physical model to facilitate testing of the ability of induction to detect low-Z metals at various depths in a tissue equivalent model.

Designing the physical model may be useful in testing the feasibility of using commercially-available inductance meters to detect the implanted tissue expander plug. This design process may be an iterative process whereby a metal detector is used to determine the location of a metal disc through simulated tissue material. The thickness of the tissue material may be increased to mimic different body types and establish the detectability of the metal as a function of tissue depth. Since detectability is a function of conductivity, it is important to establish this relationship for all metals in this investigation.

It is, therefore, possible to determine and to optimize the detectability of various low-Z alloys with an inductance meter as a function of tissue depth.

This application, therefore, provides a process by which persons having ordinary skill in the art may identify, without undue experimentation, a high puncture strength, low-Z metal that minimally disturbs the proton beam and CT images, which also can be detected within tissue by an inductance meter.

Refinement of various embodiments may further involve conducting a Virtual Clinical Trial (VCT) of the commercial vs redesigned expander using female computational phantoms for creation of proton radiotherapy plans with a predicted improvement of >10% decrease in lung V20 with the new design.

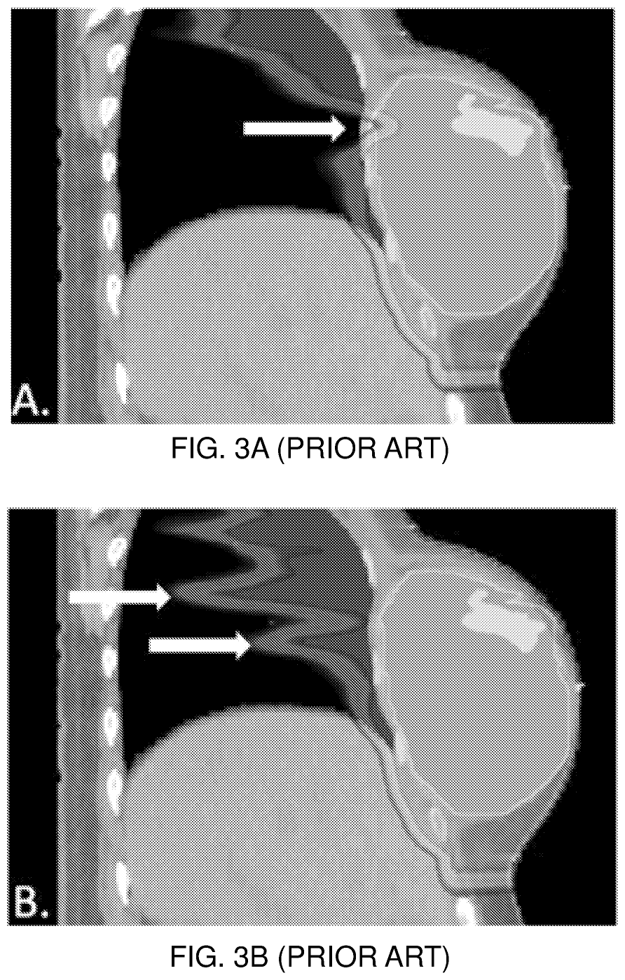

It is possible to conduct a VCT of the new breast expander design following selection of the optimal metal alloy studies. Comparisons of both CT image quality for radiotherapy treatment planning and dose-volume histograms within both targeted and non-targeted tissues delivered by proton radiotherapy may be compared between postmastectomy females with the redesigned expander and those for which the current commercially available expander is inserted. The patients for this VCT may be a series of computational anatomic phantoms based upon the reference adult female established recently by the ICRP (International Commission on Radiological Protection) Committee 2, for example, as shown in FIGS. 4A and 4B and described in detail within ICRP Publication 145. FIG. 4A is an example of a known adult female polygon mesh phantom from the International Commission on Radiological Protection (ICRP) which is useful for testing purposes as a virtual patient. FIG. 4B is an example of a cross-section of the known adult female polygon mesh phantom according to FIG. 4A. Multiple virtual patient phantoms have evolved over the past 4 decades from anatomical models based upon geometric surface equations (stylized phantoms—organ/body region boundaries constructed as spheres, ellipsoids, truncated cones, etc.) to those based upon CT or MR images (voxel phantoms—organs constructed as segmented collections of image voxels). Mesh-based phantoms, however, retain the anatomic realism of voxel phantoms while also possessing the non-uniform scalability of stylized phantoms.

### Virtual Patient Modeling

It is possible to perform whole-body scaling of the ICRP reference adult female to create three virtual patient phantoms—those at 25th, 50th, and 75th weight percentile at 50th standing height as based upon current U.S. body morphometry surveys conducted by CDC's National Center for Health Statistics (www.cdc.gov/nchs). These models will thus target total body mass, as well as various body circumferential data at these weight percentiles (e.g., abdominal girth, thigh circumference, head circumference). Furthermore, for each of these three virtual female patients, breast volumes at 10th, 25th, 50th 75th, and 90th percentiles at each total body weight will be created-again, by non-uniform sizing of the existing breast model within the three resized ICRP reference females. This will thus generate a 15-patient library of virtual patients for our VCT.

### Virtual Tissue Expanders and CT Imaging

It is possible to develop a mesh-based computer model of (1) the existing commercial expander, and (2) the redesigned expander. These expander models may then be inserted within each of the 15 virtual patients giving us a final virtual patient enrollment of 30 patients—3 weight percentiles, 5 breast sizes, and 2 expander designs. Following completion of each patient model, CT image for proton treatment planning will be created using the CT Image Projector developed by Duke University and provided to the user community as part of its newly NIBIB-funded Center for Virtual Imaging Trails (P41). The CT Image Projector will be run with the exact specifications to include the CT scanner make/model and all imaging technique factors (kVp, mAs, filter, collimation setting) used in the clinical workflow at the UFHPTI. The resulting virtual CT image sets—one for each of the 30 virtual patient phantoms. The image sets may be assessed to determine the image quality of the CT images in regards to streak artifacts using the measured HU in Aim 1C, and (2) use the CT images with the RayStation TPS at UFHPTI.

### Proton Therapy Planning

Target and OAR contours may be drawn by a radiation oncologist with the assistance of a dosimetrist. Proton therapy treatments may be designed on these simulated CT images and dose—volume histograms—both in targeted and non-targeted regions of the virtual patients—may be computed and then intercompared. The volume of ipsilateral lung receiving 20 Gy (V20) may be the primary variable for assessment. The plans with the redesigned expander may have at least a 10% reduction in ipsilateral lung V20. This pilot data may be used to power a future study. It is possible to assess the difference in dose to the ribs and chest wall target immediately posterior to the expander port. In addition, daily set-up variation may be simulated for the two expander types and the effect of set-up error on the DVH will be analyzed to assess for increase in OAR dose and/or decrease in coverage of the chest wall target.

Without wishing to be bound by theory, it is hypothesized that the redesigned expander—across each of the 15 virtual proton radiotherapy patients will result in minimal CT artifacts, will permit more optimal radiotherapy plans for these patients, will result in lower dose distributions to non-target tissues regions such as the lung, and will reduce dose perturbation due to the high-Z metal and daily set-up uncertainties, than afforded by the current commercially available device.

It is, therefore, possible without undue experimentation for a person having ordinary skill in the art with the benefit of the present disclosure to provide DVH data on 30 female phantoms of various body and breast sizes with comparative results for the commercial and the redesigned expander, providing pilot data on which to develop further studies necessary to advance this redesign.

If the proposed low-Z alloys do not meet the necessary specifications, it is possible to consider other low-Z alloys in an iterative fashion until a material that meets the specifications is identified. That said, preliminary data demonstrate minimal artifacts with the Mg alloy.

Less dose perturbation and less lung dose may also be simulated results. This will still need to be tested in real world phantoms and then patients.

### EXAMPLES

### Introduction

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for.

The purpose of the following examples is not to limit the scope of the various embodiments, but merely to provide examples illustrating specific embodiments.

### Examples 1-3

Examples 1-3 provide results for the CT images and HU profiles cross the solid water phantom 1 cm below various materials.

### Example 1

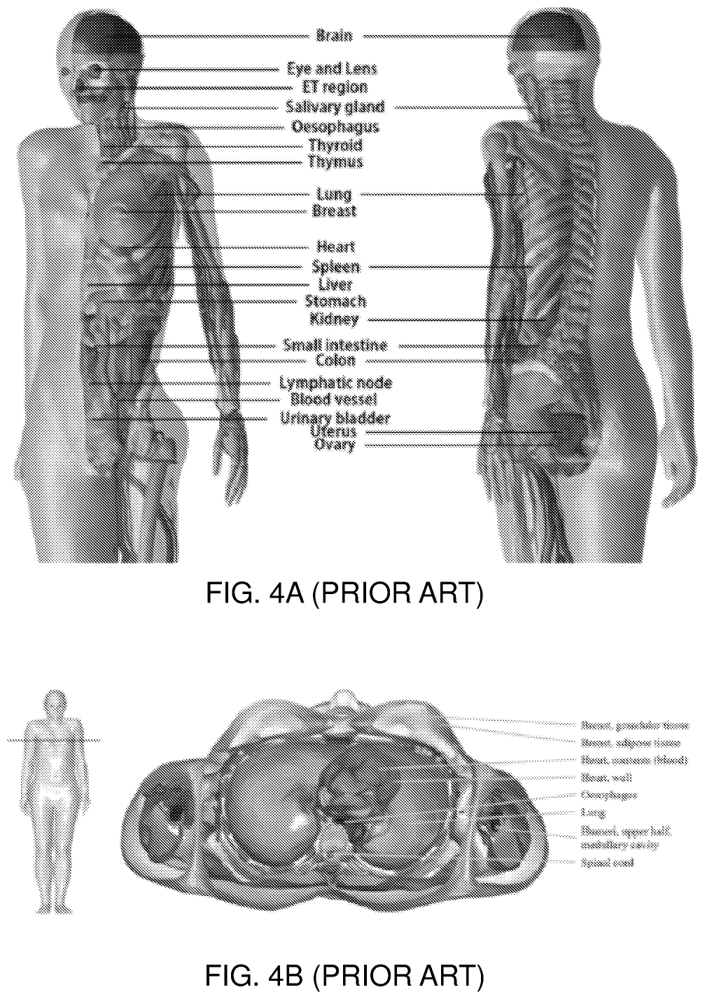

FIGS. 5A and 5B shows Nd magnet placed on the solid water phantom, resulting in observation of a streaking artifact and the HU number across the solid water reaching 1000 due to the artifact. More specifically, FIG. 5A is an example, illustrating a CT image across the solid water phantom 1 cm below a prior art tissue expander including a Nd magnet. FIG. 5B is an example according to various embodiments, illustrating a HU profile across the solid water phantom 1 cm below a prior art tissue expander including a Nd magnet.

### Example 2

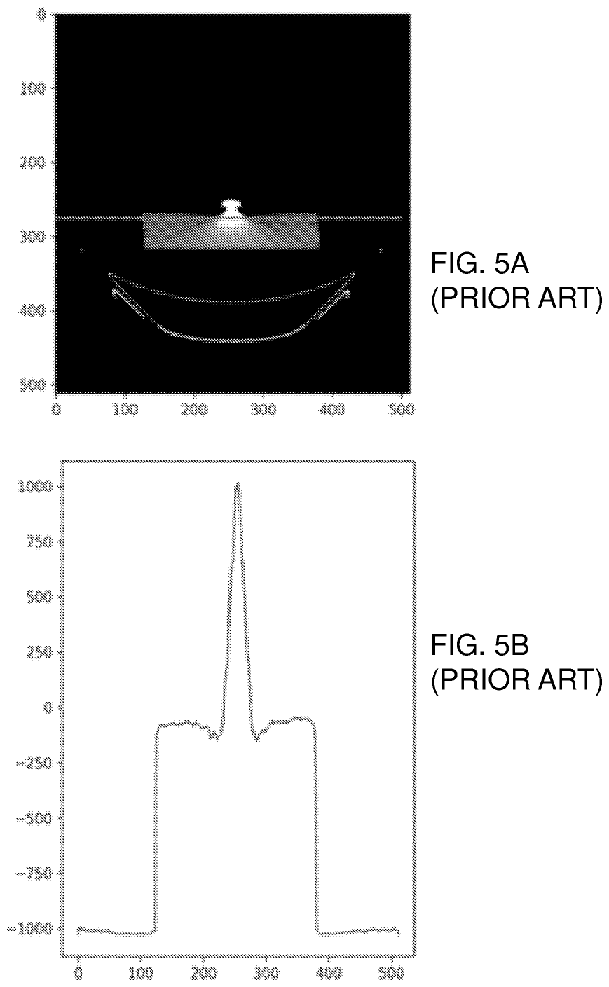

FIGS. 6A and 6B shows a Mg-alloy placed on the solid water phantom, no CT artifact is observed and the HU number remains near 0. More specifically, FIG. 6A is an example, illustrating a CT image across the solid water phantom 1 cm below a tissue expander according to various embodiments that comprises a Mg alloy. FIG. 6B is an example, illustrating a HU profile across the solid water phantom 1 cm below a tissue expander according to various embodiments that comprises a Mg alloy.

### Example 3

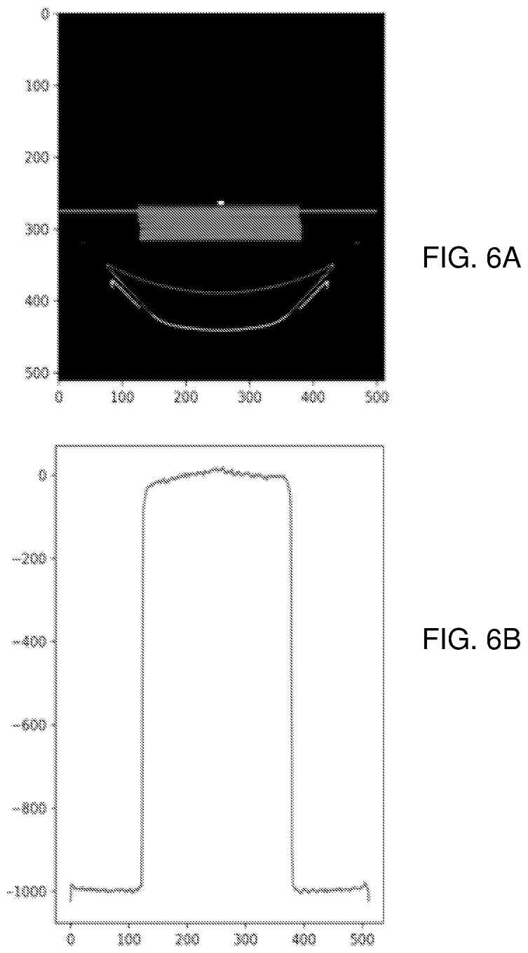

FIGS. 7A and 7B demonstrates the HU number of the solid water, which is closely approximated despite the presence of the Mg alloy as seen in in FIGS. 6A and 6B, but not by the Nd magnet as shown in FIGS. 5A and 5B. More specifically, FIG. 7A is an example, illustrating a CT image across the solid water phantom 1 cm below a tissue expander according to various embodiments that comprises no high-Z material in the field. FIG. 7B is an example according to various embodiments, illustrating a HU profile across the solid water phantom 1 cm below a tissue expander according to various embodiments that comprises no high-Z material in the field, which provides the ground truth of the HU number of the solid water phantom.

### Discussion

From this preliminary result, the high-Z magnet in the tissue expander causes an overestimation of the RSP by 56% (estimated 1.580 due to the CT artifact vs. ground truth of 1.015). The overestimated RSP results in an underestimation of the beam range in the TPS. Therefore, the organs-at-risk (OAR) downstream of the target, such as the heart and lung, will receive higher doses than that predicted by the TPS, resulting in an overdose to these adjacent OARs.

What is claimed is:

1. A port for a tissue expander, the port comprising a low-z material.

2. The port according to claim 1, wherein the low-z material comprises one or more materials selected from the group consisting of aluminum, magnesium, combinations thereof, and alloys thereof.

3. The port according to claim 2, wherein the low-z material comprises an alloy of aluminum, magnesium, or a combination thereof.

4. The port according to claim 3, wherein the alloy comprises greater than 50 percent by weight of either aluminum or magnesium.

5. The port according to claim 3, wherein the alloy further comprises an additional material selected from the group consisting of Zn, Ca, Manganese, Silicon, Yttrium, Lithium, and combinations thereof.

6. The port according to claim 5, wherein the alloy comprises from 0 to about 5 percent by weight of the additional material.

7. The port according to claim 1, wherein the tissue port has a thickness of about 0.1 to about 3 mm.

8. The port according to claim 1, wherein the tissue port is MRI and CT compatible.

9. A method for locating a port of a tissue expander implanted within a patient, the method comprising detecting the port using an inductance meter.

10. A breast tissue expander for implantation following breast surgery, the breast tissue expander comprising:
a pouch, defining a compartment adapted to hold a fluid; and
a tissue expander port, disposed on the silicone pouch and in fluid communication with the compartment to allow injection of the fluid into the compartment, and the tissue expander port comprising a low-z material, wherein the pouch is optionally comprised of silicone.

11. The breast tissue expander according to claim 10, wherein the low-Z material comprises at least one material selected from the group consisting of aluminum, magnesium, combinations thereof, and alloys thereof.

12. The breast tissue expander according to claim 10 or 11, wherein the alloy comprises greater than 50 percent by weight of either aluminum or magnesium.

13. The breast tissue expander according to any of claims 10-12, wherein the alloy further comprises an additional material selected from the group consisting of Zn, Ca, Manganese, Silicon, Yttrium, Lithium, and combinations thereof.

14. The breast tissue expander according to claim 13, wherein the alloy comprises from 0 to about 5 percent by weight of the additional material.

15. The breast tissue expander of any of claims 10-14, wherein the tissue port has a thickness of about 0.1 to about 3 mm.

16. The breast tissue expander according to any of claims 10-15, wherein the tissue port is MRI and CT compatible.

17. A system comprising a breast tissue expander according to any of claims 10-16, and an inductance meter for locating the port after implantation.

18. A method for expanding breast tissue, the method comprising:
a. implanting a breast tissue expander according to any of claims 10-16 into a patient;
b. locating the port of the breast tissue expander; and
c. injecting fluid into the pouch via the port.

19. The method of claim 18, wherein the locating the port comprises use of an inductance meter.

20. The method of claim 18, further comprising obtaining an image of the breast tissue expander upon implantation.

21. The method of claim 20, wherein obtaining image comprises use of x-ray imaging or magnetic resonance imaging.
